(19) United States
(12) Patent Application Publication
(10) Pub. No.: US 2003/0149307 A1
(43) Pub. Date: Aug. 7, 2003

(54) Process for the preparation of polyethylene glycol bis amine

(75) Inventors: Ton That Hai, Round Lake, IL (US); Larry J. Markoski, Champaign, IL (US); David E. Pereira, Apex, NC (US); Mark Nordhaus, Antioch, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Baxter International Inc.

(21) Appl. No.: 10/272,399

(22) Filed: Oct. 15, 2002

Related U.S. Application Data
(60) Provisional application No. 60/336,211, filed on Oct. 24, 2001.

(51) Int. Cl. C07C 217/28; C 07C 2 9/04

(52) U.S. Cl. 564/469; 564/505

(57) ABSTRACT

A two step process for the preparation of polyethylene glycol-bis amine comprising a first step of reacting the terminal hydroxy groups of polyethylene glycol with a halogen substituted aromatic sulfonyl halide in a solvent to form a disubstituted sulfonyl activated polyethylene glycol intermediate. In a second step the intermediate is directly aminated with ammonia to give polyethylene glycol-bis amine.

### BACKGROUND OF THE INVENTION

The present invention generally relates to the preparation of polyethylene glycol-bis amine (PEG-bis amine), which is used as an intermediate in the synthesis of crosslinking agents. More particularly, the present invention relates to a process for the preparation of high purity PEG-bis amine which is suitable for the synthesis of crosslinking agents used for the polymerization and surface modification of hemoglobin or other proteins for pharmacological use.

Hemoglobin is an essential protein bound to the red blood cells of vertebrates. Its physiological function is as a cyclic oxygen and carbon dioxide carrier whereby it transports oxygen from the lungs to the tissues, exchanges oxygen for carbon dioxide, transports carbon dioxide from the tissue to the lungs and exchanges carbon dioxide for oxygen, thus completing the respiratory cycle. When hemoglobin is outside red blood cells (i.e., extracellular hemoglobin) the hemoglobin molecule dissociates into dimers and is rapidly cleared by the kidney. Plasma retention is improved by increasing molecular size through crosslinking within and/or between hemoglobin molecules. An ideal crosslinking agent must meet several criteria which include sufficient length to prevent steric hindrance, short enough length to maintain water solubility of the bound hemoglobin matrix, nontoxicity, and an inability of causing an immunogenic reaction.

The preparation of PEG-bis amine generally comprises two reaction steps. First, the terminal hydroxyl groups of polyethylene glycol are converted to the activated form having a leaving group. The use of leaving groups, such as chloride or p-toluenesulfonate, have been reported in the literature. Second, PEG-bis amine is prepared from the activated PEG by amination via nucleophilic displacement of the leaving group. Amination by sodium azide, hydrazine and ammonium hydroxide have been reported in literature.

Davis et al., U.S. Pat. No. 4,179,337 disclose two routes for the preparation of PEG azide which is subsequently catalytically hydrogenated to yield PEG amine. The first route involves tosylation whereby PEG is dissolved in a mixture of toluene and triethylamine (TEA). p-Toluenesulfonyl chloride is added to form PEG-tosylate that precipitates out of solution at room temperature overnight. The precipitate is collected and dissolved in ethyl alcohol. Sodium azide is next added and the reaction mixture is refluxed at boiling for 36 hours to yield PEG azide. The second route comprises halogenation whereby PEG is dissolved in a mixture of toluene and TEA. Thionyl bromide is added to form PEG-bis bromine that precipitates out of solution at room temperature overnight. The precipitate is collected and dissolved in ethyl alcohol. Sodium azide is next added and the reaction mixture is refluxed at boiling for 36 hours to yield PEG-bis azide. PEG-bis azide from either route is hydrogenated in the presence of Adams catalyst to give PEG-bis amine.

Katre et al., U.S. Pat. No. 5,206,344 disclose the preparation of PEG amine in a three-step reaction sequence. First, PEG is dissolved in methylene chloride to which pyridine and p-toluenesulfonyl chloride is added. The methylene chloride is distilled off and the resultant concentrate diluted with ethyl ether to precipitate PEG-bis tosylate. Next, collected PEG-bis tosylate is dissolved in dimethylformamide, potassium phthalate is added and the reactants are heated to reflux. PEG-bis phthalate is then precipitated from the reaction filtrate by the addition of ethyl ether. Finally, PEG-bis phthalimide is dissolved in ethyl alcohol to which hydrazine hydrate is added at reflux. The resulting PEG-bis amine is then precipitated with ether and collected. To remove impurities, a purification step is repeated in triplicate whereby the PEG-bis amine is redissolved in methylene chloride, precipitated with ether, collected and washed.

De Vos and Goethals, Makromol. Chem., Rapid Commun. 6, 53-56 (1985) report the preparation of PEG ditosylate by two different routes. In the first route, PEG is dissolved in methylene chloride to which is added the base 4-dimethylaminopyridine, triethylamine and tosyl chloride. Upon evaporation of about one half of the solvent, acetone is added to precipitate PEG ditosylate. In the second route PEG is dissolved in benzene and converted to lithium alcoholate through the addition of butyllithium. Tosyl chloride is then added to prepare PEG ditosylate which is isolated by evaporation to dryness. The PEG ditosylate is then dissolved in ethyl alcohol with the solution cooled to −18° C. to precipitate finished PEG ditosylate.

Buckmann et al., Biotechnology and Applied Biochemistry 9, 258-268 (1987) report the preparation of dichloro PEG by melting PEG at 75° C. under vacuum, adding thionyl chloride, and reacting for five hours at about 68° C. The dichloro PEG is then dried under vacuum, dissolved in water and converted to PEG-bis amine by adding 25% ammonia and holding at 55° C. for 90 hours.

The prior art processes are problematic because incomplete amination yields a mixture of both di- and monosubstituted PEG. In addition, prior art processes require harsh process conditions of high temperature, high pressure, protracted cycle time, and multiple extractions and crystallizations. These processes can be inefficient, unsafe, and environmentally burdensome with regard to both emission and waste. There is a need for a process for the preparation of PEG-bis amine that yields substantially pure product, utilizes benign process conditions and minimizes environmental burden.

### SUMMARY OF THE INVENTION

Among the objects of the present invention, therefore, is the provision of a process for the preparation of polyethylene glycol-bis amine, the process comprising reacting a polyethylene glycol and a substituted aromatic sulfonyl halide, or a salt thereof, in at least one solvent, to form an intermediate, followed by amination of the intermediate to form the polyethylene glycol-bis amine.

Another embodiment of the invention is directed to a method of preparing hemoglobin crosslinking compounds comprising the use of the polyethylene glycol-bis amine prepared by the process of the invention as the polyethylene glycol-bis amine starting material.

Other objects and advantages of the invention will be apparent from the following detailed description.

### DETAILED DESCRIPTION OF THE PREFERRED EMBODIMENTS

In accordance with the present invention, it has been discovered that polyethylene glycol-bis amine, such as PEG-bis-amine [α-2-aminoethyl)-ω-aminopolyoxyethylene], may be prepared by a two step process wherein polyethylene glycol is first reacted with a halogen substituted aromatic sulfonyl halide in at least one solvent to form a PEG-bis sulfonate intermediate which is then aminated to form PEG-bis amine.

In particular, it has been discovered that the terminal hydroxy groups of polyethylene glycol can be converted to an activated form having a leaving group that will yield PEG-bis amine of greater purity than prior art processes. The use of leaving groups for amination, such as chloride or p-toluenesulfonate, has been reported in the prior art processes detailed above. However, their use requires harsh reaction conditions such as high temperature, high pressure and extended cycle times. In addition, amination in the prior art processes has been found to be incomplete resulting in unacceptably high level of PEG-mono amine. It has been discovered that a halogen, particularly fluorine, on the ring of an aromatic leaving group such as benzenesulfonyl provides a superior leaving group and increases the propensity for nucleophilic substitution. As a consequence, in contrast to prior art processes, the synthesis of PEG-bis amine may be carried out under mild conditions, in less time and with essentially complete conversion.

In one embodiment of the invention for the activation of PEG, shown in Reaction Sequence 1, PEG having an average molecular weight of about 1,000 to about 10,000 daltons is dissolved in a suitable solvent and a base is then added. The PEG solution is then reacted with a substituted aromatic sulfonyl halide. Preferred solvents include cyclic hydrocarbons, aromatic hydrocarbons and chlorinated hydrocarbons. More preferred solvents include tetrahydrofuran, dioxane, benzene, toluene, chloroform and methylene chloride. Especially preferred solvents are toluene and methylene chloride. The aromatic sulfonyl halide is substituted with an electron-withdrawing group, R, to increase the electron-withdrawing capability of the sulfonyl group. Substitution may be at one or more of the ortho, meta or para positions. Preferred R groups are halogen, nitro, fluoromethyl, difluoromethyl, trifluromethyl, substituted carboxyl, and a multi-halogen substituted benzenesulfonyl. More preferred is halogen. 4-Fluorobenzene-sulfonyl halide (4-FBSX) is an especially preferred substituted aromatic sulfonyl halide. Preferred halides, X, include chloro, fluoro or bromo with chloro most preferred. Preferred bases include n-butyllithium, tert-butyllithium and sec-butyllithium with n-butyllithium being especially preferred.

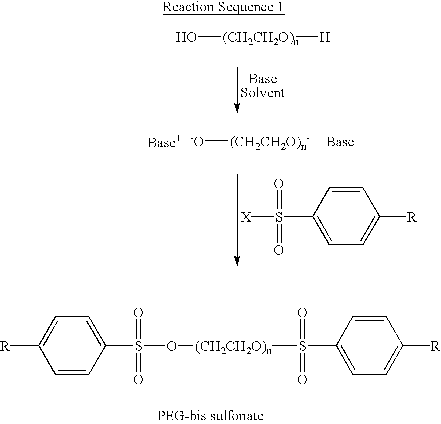

In a reaction scheme for the preparation of a preferred species, bis(4-fluorobenzenesulfonyl)PEG3400 (BFBS-PEG), PEG having an average molecular weight of about 3400 daltons (PEG-3400) is dissolved in a suitable solvent to a concentration of about 0.02 mol/L to about 0.08 mol/L and warmed to a temperature of about 25° C. to about 80° C. About 2.0 to about 5.0 mols of n-butyllithium base are added to one mol of PEG-3400. It is believed, without being bound to any particular theory, that the base reacts with the terminal PEG hydroxy groups to give the dilithio derivative. The lithium may then be quantitatively substituted. Substitution, and formation of BFBS-PEG, occurs with the addition of a stoichiometric excess of about 4.0 to about 6.0 mols of 4-fluorobenzenesulfonyl chloride (4-FBSCI). Generally 4-FBSCI is added at a rate of about 0.05 to about 0.3 stoichiometric equivalents of 4-FBSCI per minute per equivalent of PEG 3400 while maintaining the reaction temperature between about 20° C. and about 40° C. The temperature is maintained at about 25° C. for about 1 hour to about 2 hours after the 4-FBSCI addition is complete. The lithium salt is removed via solid-liquid separation, and the resulting liquor is next stripped under reduced pressure while maintaining the temperature less than about 35° C. to leave an oil containing BFBS-PEG.

The oil is dissolved in a suitable solvent with chlorinated alkyl solvents preferred. Chloroform and methylene chloride are more preferred with chloroform particularly preferred. Generally about 5 L to about 10 L of solvent per mol of starting PEG 3400 is required for oil dissolution. BFBS-PEG is precipitated by the addition of an alkyl ether at a ratio of about 10 L per L of solvent. Alkyl ethers having alkyl group lengths of five carbons or less are preferred with ethyl ether and propyl ether particularly preferred. The BFBS-PEG is then isolated by solid-liquid separation methods known to those of skill in the art, washed with additional ether and dried under high vacuum at a temperature of less than about 35° C.

In a second embodiment of the invention for the activation of PEG, shown in reaction sequence 2, PEG having an average molecular weight of about 500 to about 20,000 daltons is dissolved in at least one solvent and reacted with a halogen substituted aromatic sulfonyl halide in the presence of a base. R and X are as defined above. Preferred solvents include aromatic hydrocarbons and chlorinated hydrocarbons. More preferred solvents include benzene, toluene, chloroform and methylene chloride. Especially preferred are toluene and methylene chloride. The preferred base is selected from the group consisting of tertiary alkyl amines, with triethyl amine (TEA) especially preferred. 4-FBSCI is the preferred halogen substituted aromatic sulfonyl halide.

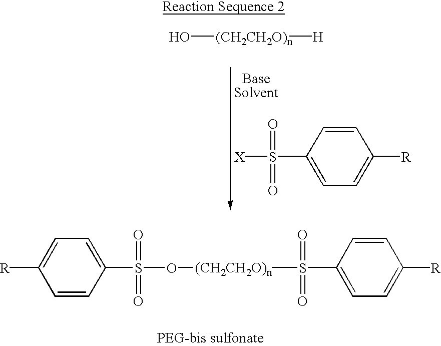

In a reaction scheme of the second embodiment for the preparation of BFBS-PEG, PEG 3400 is added to a suitable solvent to a concentration of about 0.1 mol/L to about 0.3 mol/L and warmed to a temperature of about 20° C. to about 40° C. with agitation. TEA in an amount of about 0.6 to 0.8 L per mol PEG 3400 is added with agitation. Substitution, and formation of BFBS-PEG, occurs upon 4-FBSCI addition with substantial reaction completion after about 5 hours. Ethyl acetate in an amount of about 10 L per mole PEG 3400 is next added followed by filtration and cake wash with ethyl acetate in an amount of about 3.6 L per mole PEG 3400.

The BFBS-PEG is next isolated and dried. Alkyl ether in an amount of about 100 L per mole PEG 3400 is added to the filtrate with rapid agitation whereupon the BFBS-PEG precipitates from solution to form a slurry. Alkyl ethers having alkyl groups of five carbons or less are preferred with ethyl ether and propyl ether particularly preferred. The BFBS-PEG is then collected by solid-liquid separation methods known to those of skill in the art, washed with additional ether and dried under high vacuum at a temperature of less than about 35° C.

In a third embodiment of the invention depicted in reaction sequence 3, PEG-bis amine is synthesized from PEG-bis sulfonate via nucleophilic displacement by ammonia of the substituted aromatic sulfonyl group, wherein R is defined as above, extracted from the reaction system, purified and dried. Since substitued sulfonate is a good leaving group, the amination may be performed at room temperature with quantitative yield in a short reaction time.

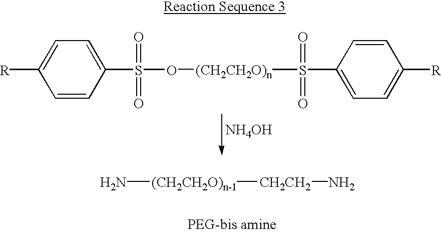

In a preferred embodiment, BFBS-PEG from either of the first two embodiments is dissolved with agitation in 29% aqueous ammonia to a concentration of between about 0.005 to 0.03 moles per liter. A suitable solvent is added to extract PEG-bis amine from the aqueous phase. Preferably, the solvent is sparingly soluble in water, and of sufficient density to easily form a two-phase system with the aqueous phase. It is also preferred that the solvent have a coefficient of distribution with PEG-bis amine such that its migration from the aqueous phase to the solvent phase is favored, and a coefficient of distribution with impurities such that their migration from the aqueous phase to the solvent phase is disfavored. Chloroform and methylene chloride are the preferred solvents with chloroform most preferred. Generally solvent to ammonium hydroxide volume ratios of about 1:2 to about 2:1 are used and the total solvent volume may be divided into multiple extractions. After extraction, the solvent containing PEG-bis amine is typically emulsified. To break the emulsion, a saturated aqueous salt solution, preferably sodium chloride, is added in a volume ratio of salt solution to solvent of about 1:2 to about 1:5 with agitation, and subsequently removed via phase separation. The solvent is then dried through the addition of a salt which is sparingly soluble in the solvent; preferably anhydrous magnesium sulfate or sodium sulfate is used. The PEG-bis amine is then reduced to an oil by azeotropic stripping of substantially all of the remaining water and solvent under high vacuum at a temperature below about 35° C. The oil is next dissolved in a volume of solvent sufficient to give a molar concentration based on BFBS-PEG of about 0.1 to about 0.4 moles per liter. PEG-bis amine is then precipitated from solvent solution by the addition of an ether, preferably ethyl ether, to a volume ratio of ether to solvent of about 50:1 to about 10:1. PEG-bis amine is collected by solid-liquid separation methods known to those of skill in the art, washed with additional ether and dried under high vacuum at a temperature of less than about 35° C.

PEG-bis amine may be used as the starting material for the synthesis of crosslinking agents used in polymerizing proteins such as hemoglobin. Preferred crosslinking agents include bismaleimide PEGs which can form an intermolecular crosslink between two hemoglobin molecules, resulting in hemoglobin dimers, trimers, tetramers, and other polymers. The bismaleimide can be prepared by various processes. For example, the preparation of dimaleimide PEG is disclosed by Tagawa et al. in EP 607978 B1 to include the dissolution of PEG-bis amine in chloroform, and dehydration by a molecular sieve, followed by addition of N-(ε-maleimidocaproyloxy) succinimide and triethylamine. Alternatively, a solution of PEG-bis amine in acetonitrile may be added to a solution of succinimidyl maleimidopropionate (SMP) in acetonitrile to form bismaleimide PEG which is then concentrated, dissolved in a salt solution, extracted with chloroform and precipitated with ethyl ether. In another method, an aqueous solution of PEG-bis amine in sodium carbonate may be added to a chilled solution of SMP in acetonitrile to form bismaleimide PEG which is then concentrated, dissolved in a salt solution, extracted with chloroform and precipitated with ethyl ether.

The following examples are presented to describe preferred embodiments and utilities of the present invention and are not meant to limit the present invention unless otherwise stated in the claims appended hereto.

### EXAMPLE 1

### Preparation of Bis(4-fluorobenzenesulfonyl)PEG3400

PEG with an average molecular weight of 3400 was dissolved in toluene with warming while under a constant nitrogen purge. After cooling to room temperature, n-butyllithium (n-BuLi) in toluene was added, dropwise, to the solution. A solution of 4-fluorobenznesulfonyl chloride (4-FBSCI) in toluene was then added, dropwise, and the reaction mixture was constantly stirred for about 1.5 hours. Next, the reaction mixture was filtered through a Celite®521 pad and the filtrate was concentrated to an oil. The oil product was dissolved in chloroform and ethyl ether was added to precipitate BFBS-PEG over the course of about 2 hours. The white solid product was collected by filtration, washed with ethyl ether (Et₂O), and dried under high vacuum pull. Analysis by NMR spectroscopy indicated the absence of impurities thus demonstrating essentially complete conversion of PEG3400 to BFBS-PEG. Table 1 below gives the quantities of reagents used and yield results for five replicate preparations. The scheme is depicted in Reaction Sequence 4.

TABLE 1

| PEG3400 | n-BuLi | 4-FBSC | CHCl3 | (Et)₂O | Yield: (1) Weight in grams; (2) |
|---|---|---|---|---|---|
| (mmol) | (mmol) | (mmol) | (mL) | (mL) | mmol^{a}; (3) % of theory^{a} |
| 60.3 | 188 | 609 | 500 | 5000 | (1) 177.4; (2) 47.7; (3) 79.1% |
| 60.3 | 188 | 607 | 500 | 5000 | (1) 170.3; (2) 45.8; (3) 76.0% |
| 60.3 | 188 | 607 | 500 | 5000 | (1) 163.7; (2) 44.1; (3) 73.1% |
| 60.3 | 193 | 609 | 500 | 5000 | (1) 168.8; (2) 45.4; (3) 75.3% |
| 60.3 | 188 | 608 | 500 | 5000 | (1) 174.1; (2) 46.9; (3) 77.8% |

Note:
^{a}These values were calculated assuming a bis(4-fluorobenzenesulfonyl)-
PEG3400 average molecular weight of 3716.

Reaction Sequence 4

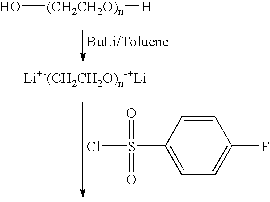

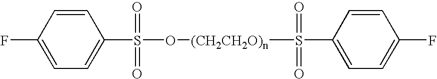

BFBS-PEG

### EXAMPLE 2

### Alternate Preparation Method for Bis(4-fluorobenzenesulfonyl)PEG3400

29.4 mmol PEG-3400 was combined with 128.4 mmol 4-FBSCI and 180 mL methylene chloride with agitation and under a nitrogen blanket. After homogeneity was achieved, 20 mL TEA was added. After approximately one-half hour a precipitate of TEA hydrochloride could be seen. The reaction was monitored for completeness by diluting small reaction aliquots with methylene chloride and testing by thin layer chromatography (TLC). The reaction was complete in 5 hours. 300 mL of ethyl acetate was then added and the slurry was filtered through a coarse glass-sintered funnel. The collected cake was washed with 100 mL ethyl acetate to give a total mother liquor volume of 580 mL. Three one-liter portions of ethyl ether were then added with rapid agitation. BFBS-PEG began to precipitate after the second ethyl ether addition, and a thick slurry resulted after the third addition. Upon agitation overnight, the BFBS-PEG was collected on a glass-sintered funnel and washed with one liter of ethyl ether. The BFBS-PEG was dried for 3 hours in a high-vacuum oven at 30° C. to give 98 grams (26.5 mmol) of BFBS-PEG (90.1% yield). NMR spectroscopy analysis showed the BFBS-PEG to contain trace amounts of TEA hydrochloride and 4-FBSCI, and no PEG-3400. The scheme is depicted in Reaction Sequence 5.

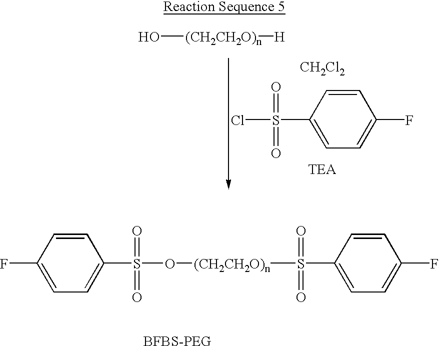

### EXAMPLE 3

### Preparation of PEG-bis Amine-3400

BFBS-PEG was dissolved in ammonium hydroxide (about 29% ammonia) and the reaction mixture was stirred constantly at room temperature overnight. The mixture was extracted with a few portions of chloroform which were combined and back-extracted once with a saturated sodium chloride solution. The organic layer was dried with anhydrous magnesium sulfate and filtered through a funnel containing additional magnesium sulfate. The filtrate was concentrated to an oil and diluted with chloroform. PEG-bis amine-3400 was precipitated overnight from agitated solution by the addition of ethyl ether. The solid PEG-bis amine-3400 was collected by filtration, washed with ethyl ether, and dried under high vacuum pull. Analysis by NMR spectroscopy analysis revealed the absence of PEG-mono amine-3400 indicating complete conversion of BFBS-PEG to PEG-bis amine-3400. Table 2 below gives the quantities of reagents used and yield results for five replicate preparations. The scheme is depicted in Reaction Sequence 6.

TABLE 2

| BFBSP | NH₄OH^{a} | # × mL | NaCl sln. | mL CHCl₃/ | Yield: (1) weight in grams; |
|---|---|---|---|---|---|
| (mmol) | (mL) | CHCl₃ | (mL) | mL ether | (2) mmol^{b}; (3) % of theory^{b} |
| 47.4 | 2000 | 3 × 500 | 500 | 350/6000 | (1) 142.6; (2) 42.0; |
| | | | | | (3) 88.6% |
| 45.6 | 2000 | 3 × 500 | 500 | 350/6000 | (1) 125.4; (2) 36.9; |
| | | | | | (3) 80.9% |
| 43 | 2000 | 5 × 500 | 500 | 300/6000 | (1) 124.5; (2) 36.6; |
| | | | | | (3) 85.1% |
| 45.4 | 2000 | 3 × 500 | 500 | 300/8000 | (1) 144.6; (2) 42.6; |
| | | | | | (3) 93.8% |
| 33.6 | 1550 | 4 × 500 | 500 | 250/8000 | (1) 111.4 (2) 32.8; |
| | | | | | (3) 97.6% |

Notes:
^{a}The NH₄OH consisted of about 29% NH₃.
^{b}These values were calculated assuming a PEG3400-bis amine average
molecular weight of 3398 daltons.

Reaction Sequence 6

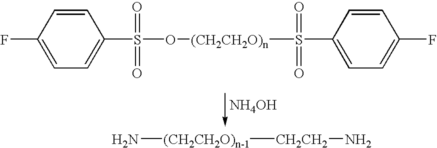

PEG3400-bis amine

### EXAMPLE 4

### Alternate Preparation Method of PEG-bis Amine 3400

The BFBS-PEG from preparative example 2 was converted to PEG-bis amine 3400 by the preparative method of example 3 with the exception that the ratio of mL ammonium hydroxide to mmol BFBS-PEG was about 80:1 and the reaction time was about 48 hours. The yield was 96 g (28.2 mmol) of PEG-bis amine-3400 (106.4% yield). The high yield resulted from excess water in the finished PEG-bis amine-3400. NMR spectroscopy analysis showed the PEG-bis amine-3400 to contain no TEA hydrochloride, 4-FBSCI or any aromatic impurity.

1. A process for the preparation of an amino polyethylene glycol, the process comprising:
reacting a polyethylene glycol and an aromatic sulfonyl halide substituted with at least one electron-withdrawing group in at least one solvent to form an intermediate; and
aminating said intermediate to form said amino polyethylene glycol.

2. The process of claim 1 wherein the amino polyethylene glycol comprises polyethylene glycol-bis amine.

3. The process of claim 1 wherein the electron-withdrawing group comprises halogen, nitro, fluoromethyl, difluoromethyl, trifluoromethyl or substituted carboxyl.

4. The process of claim 1 wherein the aromatic sulfonyl halide comprises 4-fluorobenzene sulfonyl halide.

5. The process of claim 4 wherein the 4-fluorobenzene sulfonyl halide comprises 4-fluoro benzenesulfonyl chloride.

6. The process of claim 1 wherein the intermediate comprises polyethylene glycol bis(4-fluorobenzenesulfonate).

7. The process of claim 1 wherein the polyethylene glycol has an average molecular weight of about 500 to about 20,000 daltons.

8. The process of claim 1 wherein the polyethylene glycol and the aromatic sulfonyl halide are reacted in the presence of a base.

9. The process of claim 8 wherein the base is selected from the group comprising n-butyllithium, sec-butyllithium and tert-butyllithium.

10. The process of claim 9 wherein the base comprises n-butyllithium.

11. The process of claim 1 wherein the solvent comprises a cyclic hydrocarbon, an aromatic hydrocarbon, or a chlorinated alkyl.

12. The process of claim 11 wherein the solvent comprises toluene.

13. The process of claim 1 wherein the solvent comprises toluene, the aromatic sulfonyl halide comprises 4-fluorobenzene sulfonyl chloride and the intermediate comprises polyethylene glycol bis(4-fluorobenzenesulfonate).

14. The process of claim 1 wherein the solvent comprises a chlorinated alkyl and the base comprises a trialkyl amine.

15. The process of claim 14 wherein the solvent comprises methylene chloride and the base comprises triethylamine.

16. The process of claim 15 wherein the aromatic sulfonyl halide comprises 4-fluorobenzene sulfonyl chloride and the intermediate comprises polyethylene glycol bis(4-fluorobenzenesulfonate).

17. The process of claim 1 wherein the intermediate is aminated with a nitrogen compound selected from ammonium hydroxide, hydrazine or a salt thereof, or sodium azide.

18. The process of claim 17 wherein the nitrogen compound comprises ammonium hydroxide.

19. A method of preparing hemoglobin crosslinking compounds comprising reacting the amino polyethylene glycol of claim 1 with succinimidyl maleimidopropionate.

20. The method of claim 19 wherein the hemoglobin crosslinking compound comprises bismaleimide polyethylene glycol and the amino polyethylene glycol comprises polyethylene glycol-bis amine.
